(19) United States
(12) Patent Application Publication
(10) Pub. No.: US 2024/0108354 A1
(43) Pub. Date: Apr. 4, 2024

(54) BRAIDED IMPLANT WITH INTEGRATED EMBOLIC COIL

(71) Applicant: DePuy Synthes Products, Inc., Raynham, MA (US)

(72) Inventors: Ruijiao XU, Miami Lakes, FL (US); Pedro D. PEDROSO, Raynham, MA (US)

(73) Assignee: DePuy Synthes Products, Inc., Raynham, MA (US)

(21) Appl. No.: 17/958,871

(22) Filed: Oct. 3, 2022

(51) Int. Cl. A61B 17/12

(52) U.S. Cl. CPC A61B 17/12145; A61B 17/12113; A61B 2090/3966

(57) ABSTRACT

An occlusive device can include a braided segment including an open end, a pinched end, and an embolic coil. The tubular braid can include a band positioned approximate the pinched end which can attach the embolic coil to the braided segment. In a deployed configuration, the embolic coil can extend in a distal direction from the band and form a looped shape within an approximately spherical cavity, and the braided segment can extend from the band in a distal direction and include two inversion forming three sections which at least partially overlap each other, with the pinched end affixed to an innermost section, the band affixed to the innermost section, and a middle section between the two inversion positioned within an outermost section and around the innermost section.

### FIELD OF INVENTION

The present invention generally relates to medical instruments, and more particularly, to embolic implants for aneurysm therapy.

### BACKGROUND

Cranial aneurysms can be complicated and difficult to treat due to their proximity to critical brain tissues. Recently, tubular braided implants have been introduced that have the potential to treat an aneurysm or other arterio-venous malformation easily, accurately, and safely in a parent vessel without blocking flow into perforator vessels communicating with the parent vessel. Implant devices for treating aneurysms must be delivered through long, small, tortuous blood vessels and positioning must be controlled precisely to ensure aneurysm filling without causing additional occlusions or clotting in nearby vessels. Placement of implants may further be complicated by an aneurysm being at an angle in relation to a blood vessel.

### SUMMARY

Generally, it is an object of the present invention to provide an occlusive device for occluding an approximately spherical cavity such an aneurysm sac. The device can include an embolic coil, a braided segment including an open end and a pinched end, and a band positioned approximate the pinched end attaching the embolic coil to the braided segment. In a deployed configuration, the embolic coil can extend in a distal direction from the band and can form a looped shape within the approximately spherical cavity, and the braided segment can extend in a distal direction from the band and can include two inversions. The two inversions can form three sections which at least partially overlap each other such that the pinched end is affixed to an innermost section of the three sections, the band is affixed to the innermost section of the three sections, and a middle section of the three sections extends between the two inversion and is positioned within an outermost section and around the innermost section.

In some examples, the distal end of the embolic coil can extend into the approximately spherical cavity before the outermost section of the three sections extends into the approximately spherical cavity as the occlusive device transitions from a non-deployed configuration to the deployed configuration.

In some examples, the embolic coil can include a memory shape material and the embolic coil can be effective to guide the braided segment into the approximately spherical cavity.

In some examples, the embolic coil can be positioned within a lumen of the braided segment in a non-deployed configuration.

In some examples, the occlusive device can include a microcatheter that is configured to deliver the embolic coil and the braided segment to the approximately spherical cavity in the non-deployed configuration. The microcatheter can include a lumen. The braided segment can include a diameter in the non-deployed configuration sized to fit within the lumen of the microcatheter.

In some examples, the embolic coil can be effective to facilitate a formation of the two inversions of the braided segment in the deployed configuration.

In some examples, the middle section of the three sections can be configured to provide a force pressing the outermost section of the three sections against a radial wall of the approximately spherical cavity.

In some examples, the embolic coil can be configured to provide a force pressing the outermost section of the three sections against a distal wall of the approximately spherical cavity.

In some examples, the band and the embolic coil can each include radiopaque material.

In some examples, in the deployed configuration, the open end can be positioned approximate a distal wall of the approximately spherical cavity and the band can be suspended within the approximately spherical cavity.

In some examples, the braided segment can include memory shape material, and the braided segment can include a first predetermined shape and a second deformed shape. The braided segment can be in the second, deformed shape when the occlusive device is in a non-deployed configuration, and the braided segment can move to a third, deployed shape when the occlusive device is in the deployed configuration. The third, deployed shape can be based at least in part on the predetermined shape and a shape of the approximately spherical cavity.

In another aspect, a method for constructing an occlusive device is disclosed. The method can include affixing an embolic coil to a band approximate a pinched end of a braided segment. The braided segment can include a distal open end. The occlusive device can be shaped to a predetermined shape to which the occlusive device is capable of self-expanding. Shaping the occlusive device can include forming the embolic coil into a looped shape extending in a distal direction from the band. Shaping the occlusive device can include inverting the braided segment to form a proximal inversion folded toward the distal direction thereby defining an outermost section of the braided segment. Shaping the occlusive device can include inverting the braided segment to form a distal inversion folded toward the proximal direction to thereby define a middle section between the proximal and distal inversion of the braided segment that is at least partially surrounded by the outermost section and defining an innermost section between the distal inversion and the pinched end that is at least partially surrounded by the middle section.

In some examples, the embolic coil can include a memory shape material and the embolic coil can be effective to guide the braided segment into an approximately spherical cavity. The memory shape of the embolic coil can be configured to provide an outward force to oppose the outer braid against the aneurysm wall.

In some examples, the method can include positioning the embolic coil within a lumen of the braided segment in a non-deployed configuration of the occlusive device.

In some examples, the method can include affixing the occlusive device to a locking member of a delivery system.

In another aspect, a method of occluding an approximately spherical cavity (such as an aneurysm) is disclosed. The method can include pushing an embolic coil affixed to a band positioned approximate a pinched proximal end of a braided segment of an occlusive device from a distal end of a microcatheter and into a sac of the approximately spherical cavity. The method can include positioning an embolic coil against a distal wall of the approximately spherical cavity. The method can include positioning the braided segment within the approximately spherical cavity such that the braided segment includes two inversions. The method can include positioning the band within the sac. The method can include releasing the occlusive device from a delivery system such that the braided segment and the embolic coil remain anchored within the sac.

In some examples, the method can include shaping the braided segment into three sections which at least partially overlap each other such that the pinched end is affixed to an innermost section of the three sections, the band is affixed to the innermost section, and a middle section of the three sections extends between the two inversions and is positioned within an outermost section and around the innermost section. The method can include extending a distal end of the embolic coil into the sac before the outermost section of the three sections extends into the sac.

In some examples, the method can include providing a force by the middle section that presses the outermost section of the three sections against a radial wall of the sac.

In some examples, the method can include providing a force by the embolic coil that presses the outermost section of the three sections against a distal wall of the sac.

In some examples, the method can include positioning the open end approximate a distal wall of the sac and suspending the band within the sac.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIGS. 1A-1I are illustrations of an occlusive device having a tubular braid and integrated embolic coil that expands to a predetermined shape as the tubular braid exits a microcatheter according to aspects of the present invention;

FIGS. 2A-2E are illustrations of an occlusive device similar to the implant illustrated in FIGS. 1A-I as it occludes an approximately spherical cavity, according to aspects of the present invention;

FIGS. 3A and 3B are illustrations of measurements of an example occlusive device and an approximately spherical cavity, according to aspects of the present invention;

FIGS. 4A and 4B are illustrations of an example occlusive device attached to a delivery system, according to aspects of the present invention;

FIGS. 5A and 5B are illustrations of a detachment sequence of the example occlusive device from the delivery system, according to aspects of the present invention;

FIG. 6 is a flowchart of a method for constructing an occlusive device, according to aspects of the present invention; and

FIG. 7 is a flowchart of a method for occluding an approximately spherical cavity, according to aspects of the present invention.

FIG. 8 is an illustration of an embolic coil having a branched structure according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g. “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

Examples presented herein generally include an implant including a tubular braid and an embolic coil. The implant can be configured to secure within an aneurysm sac and occlude a majority of the aneurysm's neck. The tubular braid can be set into a predetermined shape, compressed for delivery through a microcatheter, and implanted in at least one implanted position that is based on the predetermined shape and the geometry of the aneurysm in which the braid is implanted. The braid can have features similar to as described in U.S. Pat. No. 10,653,425, the entirety of which is incorporated herein by reference as if included in full, and variations thereof as understood by a person skilled in the pertinent art. In some applications, the implant may be better suited for implantation into an aneurysm that is off angle from the connected artery compared to an implant having a similar braid but lacking an embolic coil.

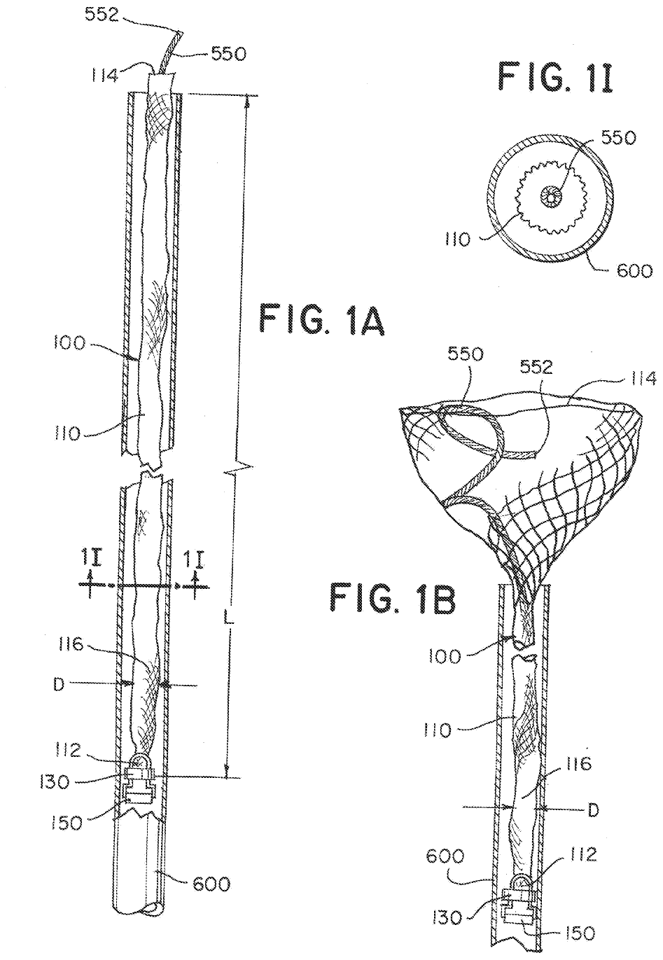

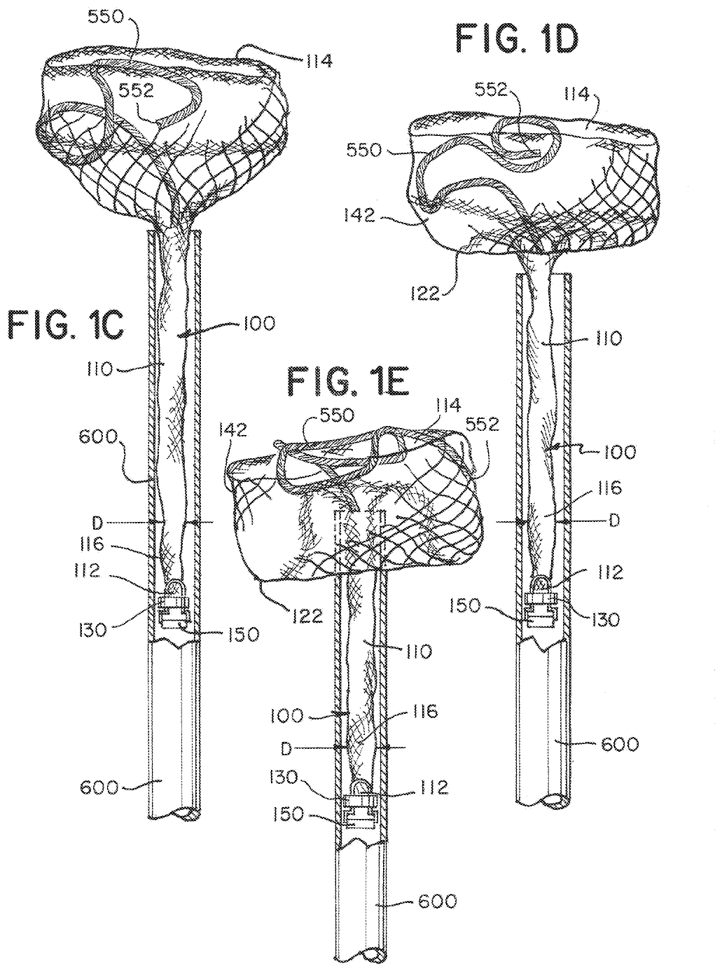

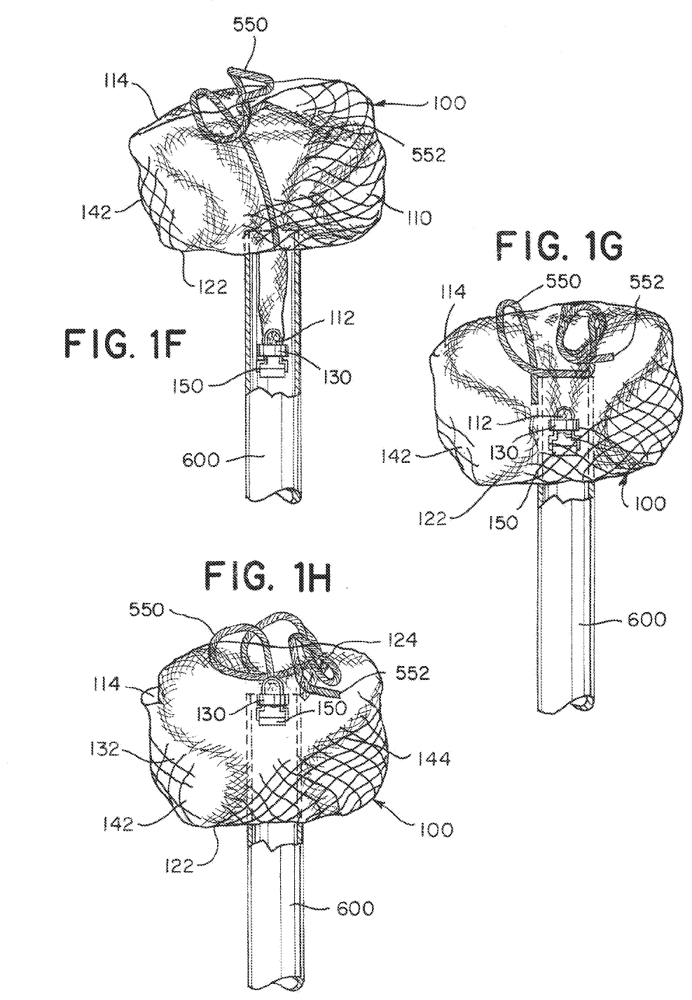

FIGS. 1A through 1I are illustrations of an example occlusive device 100 having a braid 110 expanding to a predetermined shape as the braid 110 exits a microcatheter 600. The implant 100 has a predetermined shape when deployed as illustrated in FIG. 1H.

As illustrated in FIG. 1A, the braid 110 can be shaped to a delivery shape that is extended to a single layer of tubular braid having a compressed diameter D sized to be delivered through the microcatheter 600 and a length L. The compressed diameter D can be sized to fit within a microcatheter lumen having an inner diameter of about 0.017 to about 0.032 inches. As shown, a distal end 552 of the embolic coil 550 can extend out of the open end 114 of the braid 110. The illustrated braid 110 can have a length L of about 22 mm to about 25 mm, although in some examples, the length L can be approximately 10 mm to approximately 40 mm. The embolic coil 550 can have a length of about 1 cm to about 50 cm. As will be appreciated and understood by a person skilled in the pertinent art, the length L of a specific braid 110 and length of embolic coil 550 can be tailored based on the size and shape of the aneurysm being treated. The braid 110 can include an open end 114, a pinched end 112 attached to a detachment feature 150, a radiopaque band 130 approximate the pinched end, a lumen 116 running through the tubular braid 110, and an embolic coil 550 can extend through lumen 116 and can be attached to the band 130. FIG. 1I is a cross-sectional illustration of the implant 100 and microcatheter 600 as indicated in FIG. 1A. The embolic coil 550 can be approximately concentric to the braid 110 during delivery as illustrated in FIG. 1I.

During delivery through the microcatheter 600, the detachment feature 150 can be attached to a delivery system at a proximal end of the implant 100, the pinched end 112 can be positioned near the proximal end of the implant 100, and the distal end 552 of the embolic coil 550 can define the distal end of the implant 100. Collapsing the braid 110 to a single layer tube can result in a braid 110 that has a sufficiently small diameter and a sufficiently short length L to mitigate effects of friction force on the braid 110 when it is delivered through the microcatheter, allowing the braid 110 to be delivered unsheathed in some applications. As illustrated in FIG. 1A, a distal end 552 of embolic coil 550 can be positioned to exit microcatheter 600 before any portion of the braid 110 exits the microcatheter.

As illustrated in FIG. 1B, the open end 114 can expand as it exits the microcatheter 600. If the open end 114 is unconstrained by an aneurysm as illustrated, the open end 114 can expand to its circumference in the predetermined shape. The embolic coil 550 can begin to coil within the braid lumen 116 near the open end 114, thereby facilitating the expansion of the open end 114.

As illustrated in FIG. 1C, the distal portion of the braid 110 can continue to expand radially as it exits the microcatheter 600 and the embolic coil 550 can continue to loop within the braid near the open end 114.

As illustrated in FIG. 1D, the braid 110 can form an inversion 122 defining an outer segment 142 as the braid 110 is further pushed out of the microcatheter 600. The embolic coil 550 can facilitate inversion by preventing the braid 110 from kinking or bending as the as the outer segment 142 begins to form.

As illustrated in FIGS. 1E, 1F, and 1G, the “S” shape of a middle segment 144 can begin to form as the braid 110 is further pushed from the microcatheter 600 while the embolic coil 550 facilitates the inversions formed between outer segment 142 and middle segment 144.

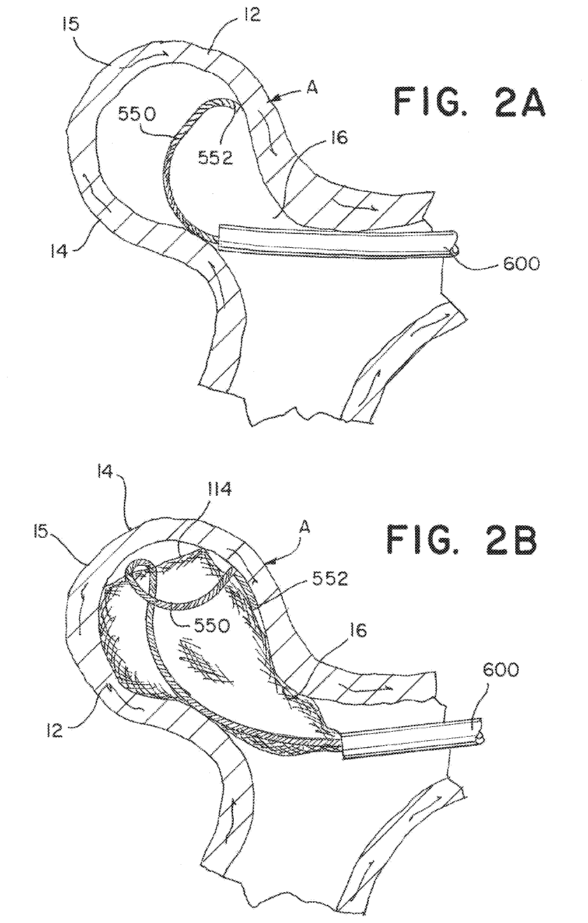

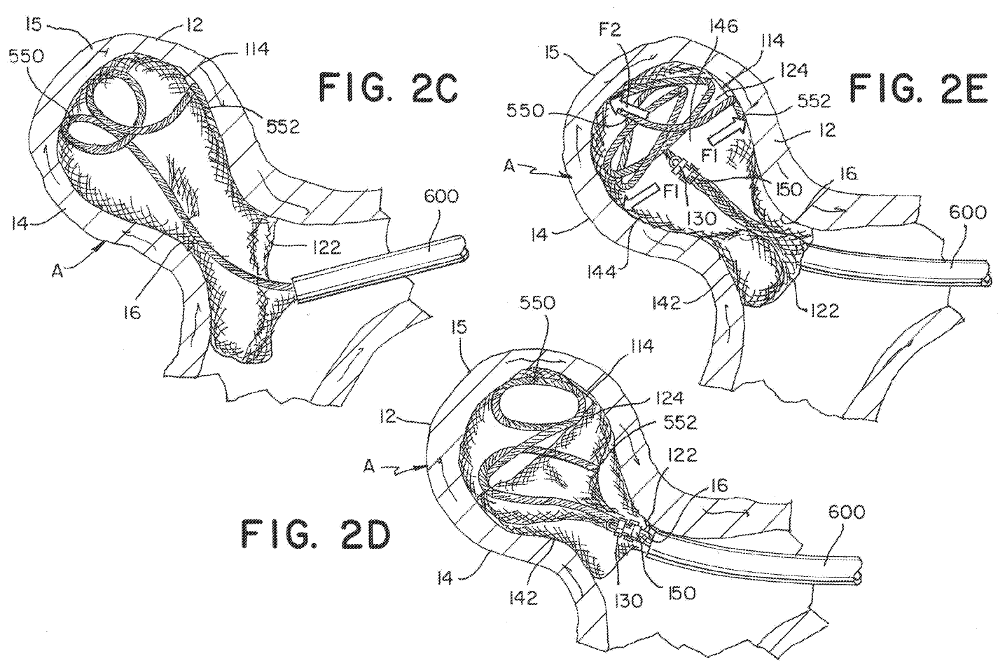

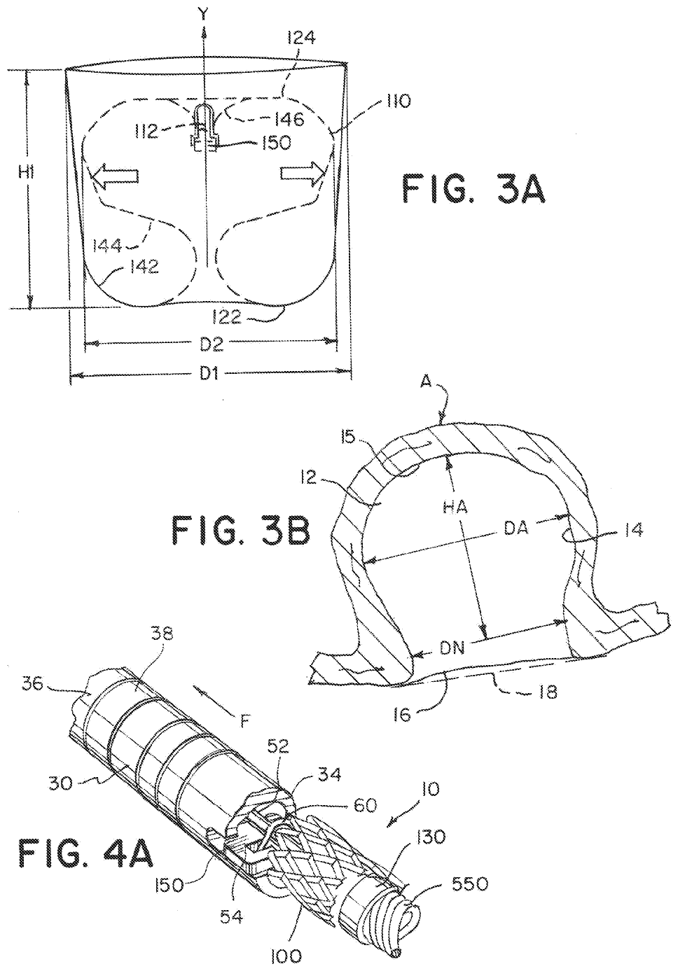

As illustrated in FIG. 1H, when all, or nearly all of the braid 110 exits the microcatheter 600, the braid 110, not confined by an aneurysm, can expand to a predetermined shape similar to the shape illustrated in FIG. 2E and FIG. 3A. In the predetermined shape, the braid 110 of the illustrated implant can have a diameter between about 6 mm and about 6.5 mm and a height between about 5 mm and about 5.5 mm. The embolic coil 550 is looped at a distal end of the implant 100 when the braid 110 is in the predetermined shape.

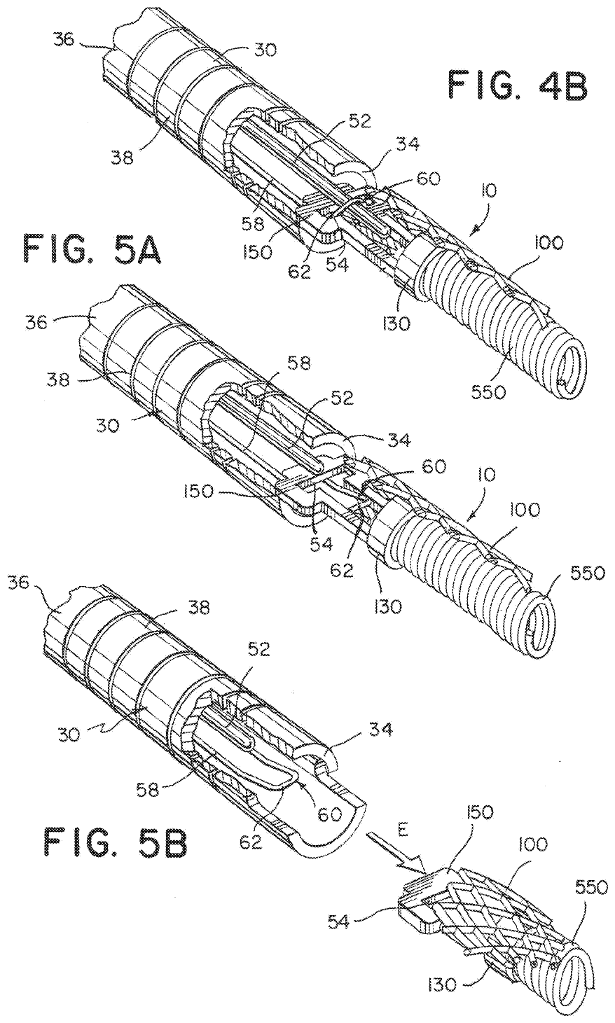

In some examples, the ratio of the outermost diameter of the braid 110 in the predetermined shape illustrated in FIG. 1H to the length of the braid 110 in the delivery shape illustrated in FIG. 1A can be between about 0.3 and about 0.24. As shown in FIG. 1H, in the predetermined shape, the tubular braid 110 can include two inversions 122, 124 dividing the braid 110 into three segments 142, 144, 146. In the predetermined shape, the braid 110 can have an outer segment 142 extending from the open end 114 of the braid 110 to one of the inversions 122, an inner segment 146 extending from the pinched end 112 of the braid 110 to the other of the inversions 124, and a middle segment 144 extending between the two inversions 122, 124. When in the predetermined shape, the tubular braid 110 can be substantially radially symmetrical about a central vertical axis y (see FIG. 3A). Detachment feature 150 can be a flat key that can be used with a mechanical implant delivery system (see FIGS. 4A-4B and 5A-5B). The middle segment 144 can have one or more bends 132, 134. The bends 132, 134 can be positioned facilitate the movement of the braid 110 into an implanted shape as illustrated in FIGS. 2D-2E and the bends 132, 134 can be positioned to stabilize the braid 110 in the implanted shape.

FIGS. 2A-2E are illustrations of an occlusive device similar to the implant illustrated in FIGS. 1A-H as it occludes an approximately spherical cavity. As shown in FIG. 2A, the microcatheter can be positioned through vasculature at a neck 16 of an approximately spherical cavity A, which may be a patient aneurysm. The approximately spherical cavity A can have a sac 12 that has a wall 14 with a distal wall portion 15 at the distal end of the sac 12. At a proximal end of the sac 12 the approximately spherical cavity A the neck 16 can connect the approximately spherical cavity A to the vasculature. As shown in FIG. 2A, the neck 16 is at an offset angle from the vasculature such that the distal end of the microcatheter is angled toward a distal edge of the neck 16, which can make it difficult to deploy braid 110. As shown, the embolic coil 550 can enter the sac 12 before any portion of the tubular braid 110 enters into the sac 12. Once embolic coil 550 enters the sac 12, it can facilitate the delivery of the tubular braid 110 over the embolic coil 550 and into the sac 12 so that the braid 110 does not unintentionally deploy into the vasculature or at an undesirable angle within the sac 12.

In FIG. 2B, the open end 114 of the tubular braid has begun to expand into the sac 12 of the approximately spherical cavity A, and the embolic coil 550 has begun to coil against the distal wall portion 15 of the wall 14.

In FIG. 2C, the embolic coil 550 continues to coil against the distal wall portion 15 of the wall 14, and the braid 110 begins to invert and form proximal inversion 122. In some examples, the coil can be shaped into a loop larger than a diameter of the approximately spherical cavity A which may facilitate the inversion of the braid 110 by pushing the sides of the braid 110 against the wall 14 of the sac to hold the braid 110 in place as it inverts to form proximal inversion 122.

In FIG. 2D, the braid 110 begins to form distal inversion 124 and the embolic coil 550 begins to occupy more volume within the sac 12. In FIG. 2D, the braid 110 has formed the outermost segment 142, middle segment 144, and inner segment 146. As shown, detachment feature 150 is suspended approximate the neck 16 of the sac 12. FIG. 2D is a fully deployed configuration, in some examples. In FIG. 2E the detachment feature 150 is pushed further distally into the sac 12 of the approximately spherical cavity A. FIG. 2E shows another deployed configuration of the braid 110. In either configuration (FIGS. 2D and 2E), the embolic coil 550 can press against the outermost section 142 of the braid 110 and against a distal wall portion 15 of the wall 14, thereby securing the occlusive device 100 within the approximately spherical cavity A. Similarly, the middle section 144 is configured to provide a force against the outermost section 142 and against the wall 14 of the sac 12. As illustrated in FIG. 2E, the middle section 144 can provide a force F1 that presses the outermost section 142 of the three sections against a radial wall of the sac 12, and the embolic coil 550 can provide a force F2 that presses the outermost section 142 of the three sections against a distal wall of the sac 12.

FIG. 3A is an illustration of height HI and diameter D1, D2 measurements of an example implant 100 in a predetermined shape. In the predetermined shape, the braid 110 of the example occlusive device 100 can be substantially radially symmetrical about vertical axis y, and therefore can have substantially circular concentric cross-sections each describable by its diameter. FIG. 3A highlights the height HI of the implant 100 in a predetermined shape measured between the inversions 122, 124, the outer diameter D1 of the outer segment 142, which corresponds to the diameter of the open end 114, and the outer diameter D2 of the middle segment D2. Although FIG. 3A illustrates only one example predetermined shape, it should be understood that the height and diameter of example occlusive devices described herein 100 and portions thereof can be measured similarly to as illustrated in FIG. 3A.

In some examples, the occlusive device 100 can rely on a radial outward force to anchor the occlusive device within the sac of an approximately spherical cavity A. To this end, the tubular braid 110 can be shaped to a predetermined shape having a diameter D1 that is greater than its height so that the braid is radially constricted when implanted in an aneurysm. The ratio of diameter to height of the braid 110 in a respective predetermined shape can be within the range of 2:1 to 1:3 to treat aneurysms of many known sizes and shapes.

FIG. 3B is an illustration of height HA, sac diameter DA, and neck diameter DN measurements of an approximately spherical cavity A. The location of the plane 18 defining a boundary between the approximately spherical cavity A and blood vessels is also illustrated. The coil 550 can have a predetermined shape that includes a looped shape that is larger in diameter than the diameter DA of the approximately spherical cavity A to provide more outward force to appose the braid 110 to the approximately spherical cavity A and facilitate the inversion. The distal open end 114 can be positioned distal of the distal inversion 124 when the braid 110 is in the predetermined shape as illustrated in FIG. 3A, or can be positioned between the proximal inversion 122 and the distal inversion 124 such as illustrated in FIG. 1H. The distal open end 114 can be shaped to a height to provide enough braid apposition and also short enough so the device 100 can fit within a short approximately spherical cavity having a shorter height HA compared to the sac diameter DA.

FIGS. 4A and 4B are illustrations of an example occlusive device attached to a delivery system 30. A delivery system 30 is attached to an occlusive device 100 via a pull wire 52, loop wire 58, and locking portion 54 of detachment feature 150. The delivery system 30 can include a distal end 34, proximal end 36, and compressible portion 38. The detachment feature 150 can be positioned just distal of the delivery system 30 and a proximal end of the occlusive device 100, 200 can be attached to a distal end of the detachment feature 150. A pull wire 52 can be positioned within a lumen of the delivery system 30. A loop wire 58 can be attached to the delivery system 30 and can include a loop opening 60 at a distal end 34 of the delivery system 30. As shown more clearly in FIG. 4B, which is a cutaway view of the delivery system of FIG. 4A, the loop opening 60 can be passed through the locking portion 54 of the detachment mechanism 150, and a distal end of the pull wire 52 can be positioned through the loop opening 60 of the loop wire 58 after it is passed through the locking portion 54. Accordingly, the detachment feature 150 can thus be attached to the distal end 34 of the delivery system 30. The compressible portion 38 can be formed of spiral cuts within the material of the delivery system 30, such that the compressible portion 38 can be compressed prior to attaching the detachment mechanism 150 to the delivery system 30. When the occlusive device 100 is detached from the delivery system, the compressible portion 38 can be configured to expand and impart an elastic force to “push” the occlusive device 100 away from the deployment tube 30 and towards the treatment site.

FIGS. 5A and 5B are illustrations of a detachment sequence of the example occlusive device from the delivery system 30. In FIG. 5A, the pull wire 52 is shown being pulled proximally towards proximal end 36 of the delivery system 30. This allows distal end of pull wire 52 to exit the loop opening 60 of loop wire 58, thereby allowing loop opening 60 to exit the locking portion 54 of detachment feature 150. FIG. 5A shows loop opening distal end 62 of loop opening exiting the locking portion 154 of detachment feature 150. In FIG. 5B, the compressible portion 38 of delivery system 30 can decompress, thereby imparting elastic force “E” to the detachment feature 150.

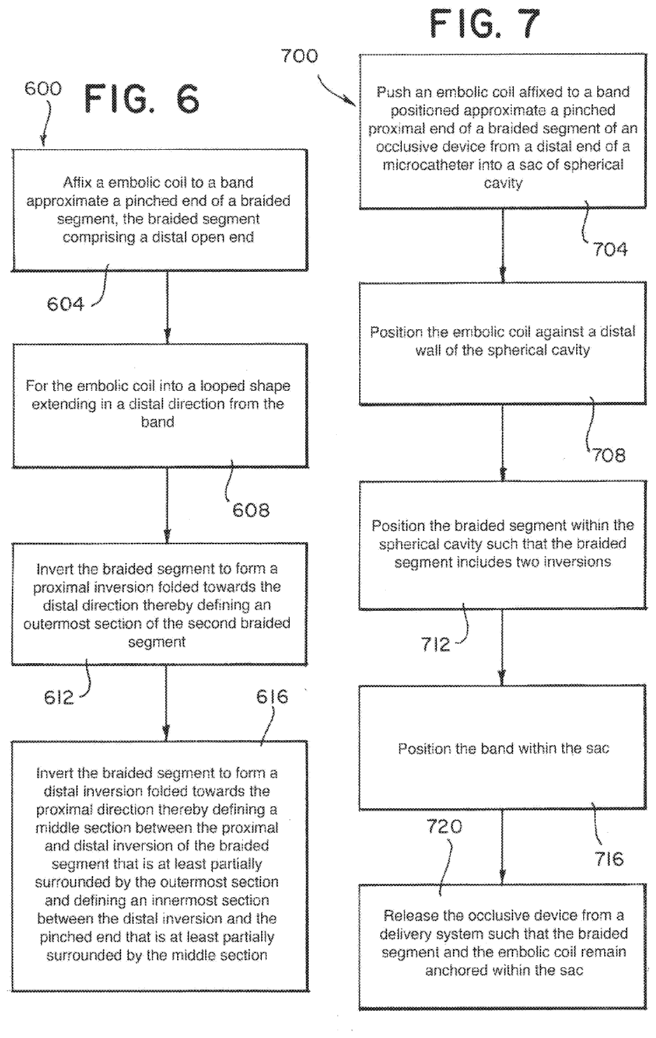

FIG. 6 is a flowchart of a method 600 for constructing an occlusive device. In block 604, the method can include affixing an embolic coil 550 to a band 130 approximate a pinched end 112 of a braided segment 110. The braided segment can also include a distal open end 114. The method can include shaping the occlusive device 100 to a predetermined shape to which the occlusive device 100 is capable of self-expanding.

In block 608, shaping the occlusive device 100 can include forming the embolic coil 550 into a looped shape extending in a distal direction from the band.

In block 612, shaping the occlusive device 100 can include inverting the braided segment 100 to form a proximal inversion 122 folded towards the distal direction to thereby define an outermost section 142 of the braided segment 110.

In block 616, shaping the occlusive device 100 can include inverting the braided segment 110 to form a distal inversion 124 that is folded toward the proximal direction to thereby define a middle section 144 between the proximal inversion 122 and the distal inversion 124 of the braided segment 110. The middle section 144 can be at least partially surrounded by the outermost section 142 and can define an innermost section 146 between the distal inversion 124 and the pinched end 112 that is at least partially surrounded by the middle section 144.

In some examples, the embolic coil 550 can be constructed of a memory shape material. The embolic coil 550 can be effective to guide the braided segment into an approximately spherical cavity A.

In some examples, the method can include positioning the embolic coil 550 within a lumen 116 of the braided segment in a non-deployed configuration of the occlusive device 100.

In some examples, the method can further include affixing the occlusive device 100 to a locking member 150 of a delivery system 30.

FIG. 7 is a flowchart of a method 700 for occluding an approximately spherical cavity. In block 704, the method can include pushing an embolic coil 550 that is affixed to a band 130 positioned approximate a pinched proximal end 112 of a braided segment 110 of an occlusive device 100 from a distal end of a microcatheter 600 and into a sac 12 of the approximately spherical cavity A.

In block 708, the method can include positioning the embolic coil 550 against a distal wall 15 of the approximately spherical cavity A.

In block 712, the method can include positioning the braided segment 110 within the approximately spherical cavity A such that the braided segment includes two inversions 122, 124.

In block 716, the method can include positioning the band within the sac 12.

In block 720, the method can include releasing the occlusive device 100 from a delivery system such that the braided segment 110 and the embolic coil 550 remain anchored within the sac 12.

In some examples, the method can include shaping the braided segment to include three sections 142, 144, 146 which at least partially overlap each other such that the pinched end 112 is affixed to an innermost section 146 of the three sections, the band is affixed to the innermost section 146 of the three sections, and a middle section 144 of the three sections extends between two inversion and is positioned within an outermost section 152 and around the innermost section 146. The method may include extending a distal end 552 of the embolic coil 550 into the sac 12 before the outermost section of the three sections extends into the sac 12.

In some examples, the method may further include providing a force F1 by the middle section 144 that presses the outermost section 142 of the three sections against a radial wall of the sac 12.

In some examples, the method may further include providing a force F2 by the embolic coil 550 that presses the outermost section 142 of the three sections against a distal wall of the sac 12.

In some examples, the method may further include positioning the open end 114 approximate a distal wall of the sac 12 and suspending the band 130 within the sac 12.

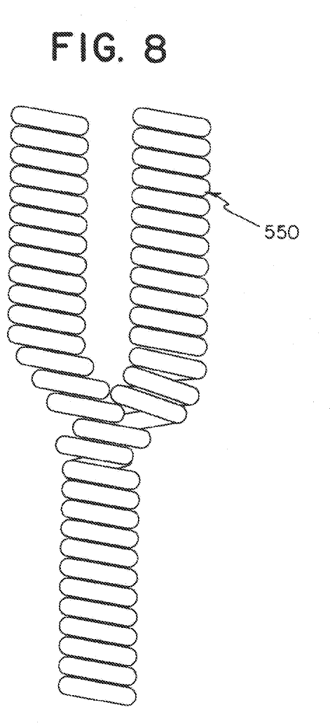

FIG. 8 is an illustration of an embolic coil 550 having a branched structure with a proximal trunk and distal branches. For the sake of illustration, the embolic coil 550 illustrated in FIGS. 1A through 5B includes a single winding coil similar to embolic coils presently used for aneurysm treatment. The examples illustrated in FIGS. 1A through 5B, and variations thereof disclosed herein and understood by a person skilled in the pertinent art, can be modified to include a more complex embolic coil structure such as illustrated in FIG. 8, can be modified to include more than two branches, and/or can be modified to include more than one coil winding attached at the detachment feature 150 as non-limiting examples.

The descriptions contained herein are examples of embodiments of the invention and are not intended in any way to limit the scope of the invention. As described herein, the invention contemplates many variations and modifications of the occlusive device, including alternative materials, alternative geometries, alternative detachment features, alternative delivery systems, alternative means for forming a braid into a predetermined shape, alternative treatment methods, etc. These modifications would be apparent to those having ordinary skill in the art to which this invention relates and are intended to be within the scope of the claims which follow.

What is claimed is:

1. An occlusive device for occluding an approximately spherical cavity, comprising:
an embolic coil;
a braided segment comprising an open end and a pinched end; and
a band positioned approximate the pinched end attaching the embolic coil to the braided segment,
wherein in a deployed configuration, the embolic coil extends in a distal direction from the band and forms a looped shape within the approximately spherical cavity and the braided segment extends in a distal direction from the band and comprises two inversions forming three sections which at least partially overlap each other such that the pinched end is affixed to an innermost section of the three sections, the band is affixed to the innermost section of the three sections, and a middle section of the three sections extends between the two inversions and is positioned within an outermost section and around the innermost section.

2. The occlusive device of claim 1, wherein the distal end of the embolic coil extends into the approximately spherical cavity before the outermost section of the three sections extends into the approximately spherical cavity as the occlusive device transitions from a non-deployed configuration to the deployed configuration.

3. The occlusive device of claim 1, wherein the embolic coil comprises a memory shape material and the embolic coil is effective to guide the braided segment into the approximately spherical cavity.

4. The occlusive device of claim 1, wherein the embolic coil is positioned within a lumen of the braided segment in a non-deployed configuration.

5. The occlusive device of claim 4, further comprising:
a microcatheter configured to deliver the embolic coil and the braided segment to the approximately spherical cavity in the non-deployed configuration, the microcatheter comprising a lumen,
wherein the braided segment comprises a diameter in the non-deployed configuration sized to fit within the lumen of the microcatheter.

6. The occlusive device of claim 1, wherein the embolic coil is effective to facilitate a formation of the two inversions of the braided segment in the deployed configuration.

7. The occlusive device of claim 1, wherein the middle section of the three sections is configured to provide a force pressing the outermost section of the three sections against a radial wall of the approximately spherical cavity.

8. The occlusive device of claim 1, wherein the embolic coil is configured to provide a force pressing the outermost section of the three sections against a distal wall of the approximately spherical cavity.

9. The occlusive device of claim 1, wherein the band and the embolic coil each comprise radiopaque material.

10. The occlusive device of claim 1, wherein, in the deployed configuration, the open end is positioned approximate a distal wall of the approximately spherical cavity, and the band is suspended within the approximately spherical cavity.

11. The occlusive device of claim 1, wherein the braided segment comprises a memory shape material, the braided segment comprising a first, predetermined shape and a second, deformed shape,
wherein the braided segment is in the second, deformed shape when the occlusive device is in a non-deployed configuration, and
wherein the braided segment moves to a third, deployed shape when the occlusive device is in the deployed configuration, the third, deployed shape being based at least in part on the predetermined shape and a shape of the approximately spherical cavity.

12. A method for constructing an occlusive device, the method comprising:
affixing an embolic coil to a band approximate a pinched end of a braided segment, the braided segment comprising a distal open end; and
shaping, as follows, the occlusive device to a predetermined shape to which the occlusive device is capable of self-expanding:
forming the embolic coil into a looped shape extending in a distal direction from the band;
inverting the braided segment to form a proximal inversion folded toward the distal direction thereby defining an outermost section of the braided segment; and
inverting the braided segment to form a distal inversion folded toward the proximal direction thereby defining a middle section between the proximal and distal inversions of the braided segment that is at least partially surrounded by the outermost section and defining an innermost section between the distal inversion and the pinched end that is at least partially surrounded by the middle section.

13. The method of claim 12, wherein the embolic coil comprises a memory shape material and the embolic coil is effective to guide the braided segment into an approximately spherical cavity.

14. The method of claim 12, further comprising positioning the embolic coil within a lumen of the braided segment in a non-deployed configuration of the occlusive device.

15. The method of claim 12, further comprising affixing the occlusive device to a locking member of a delivery system.

16. A method of occluding an approximately spherical cavity, the method comprising:
pushing an embolic coil affixed to a band positioned approximate a pinched proximal end of a braided segment of an occlusive device from a distal end of a microcatheter into a sac of the approximately spherical cavity;
positioning the embolic coil against a distal wall of the approximately spherical cavity;
positioning the braided segment within the approximately spherical cavity such that the braided segment includes two inversions;
positioning the band within the sac; and
releasing the occlusive device from a delivery system such that the braided segment and the embolic coil remain anchored within the sac.

17. The method of claim 16, further comprising:
shaping the braided segment to include three sections which at least partially overlap each other such that the pinched end is affixed to an innermost section of the three sections, the band is affixed to the innermost section of the three sections, and a middle section of the three sections extends between the two inversions and is positioned within an outermost section and around the innermost section; and
extending a distal end of the embolic coil into the sac before the outermost section of the three sections extends into the sac.

18. The method of claim 17, further comprising providing a force by the middle section that presses the outermost section of the three sections against a radial wall of the sac.

19. The method of claim 17, further comprising providing a force by the embolic coil that presses the outermost section of the three sections against a distal wall of the sac.

20. The method of claim 16, further comprising:
positioning an open end of the braided segment approximate a distal wall of the sac; and
suspending the band within the sac.
